(19) United States
(12) Patent Application Publication
(10) Pub. No.: US 2026/0348937 A1
(43) Pub. Date: Nov. 5, 2026

(54) Monitoring And Predicting Pipeline Gelation Risk

(71) Applicant: Chevron U.S.A. Inc., San Ramon, CA (US)

(72) Inventor: Joseph E. Patterson, Houston, TX (US)

(21) Appl. No.: 19/115,653

(22) PCT Filed: Oct. 30, 2023
(86) PCT No.: PCT/US2023/078257

§ 371 Date: Mar. 26, 2025

Related U.S. Application Data
(60) Provisional application No. 63/381,667, filed on Oct. 31, 2022.

(51) Int. Cl. G01N 33/28; G01W 1/10; G06F 30/20

(52) U.S. Cl. CPC G01N 33/28; G01W 1/10; G06F 30/20

(57) ABSTRACT

A computing system, method, and computer-readable medium includes a gelation analytics tool for managing a risk of pipeline gelation. The gelation analytics tool generates a wax precipitation model over a range of temperatures for batches of crude oil. The gelation analytics tool generates a pipeline temperature model of predicted temperatures along the pipeline based upon forecasted ambient conditions. The gelation analytics tool receives a proposed schedule that includes at least one shutdown of the pipeline during a selected time period and generates a wax precipitation prediction for a batch of crude oil during the selected time period by applying the pipeline temperature model to the wax precipitation model.

### RELATED APPLICATIONS

The present application claims priority to U.S. Provisional Patent Application No. 63/381,667 filed Oct. 31, 2022, the entire content of which is incorporated herein by reference.

### TECHNICAL FIELD

Embodiments of the technology relate generally to monitoring and predicting gelation risk in a hydrocarbon pipeline.

### BACKGROUND

Pipelines are used to transport hydrocarbons over long distances and in a variety of environments. In some instances, the pipelines transport hydrocarbons through extremely cold environments, including polar regions, mountainous regions, and subsea regions. In certain cases, the oil passing through the pipeline can contain a substantial wax component. When subjected to extremely cold environments, wax in the oil can solidify causing (i) viscous, non-Newtonian flow, (ii) restricted flow, and (iii) in some cases, blockages that completely disrupt the flow of oil through the pipeline. When a pipeline becomes blocked by solidified wax, it is referred to as gelation.

The pipeline gelation problem often occurs during a shutdown of the pipeline. For example, a pipeline may be shutdown to perform planned or unplanned maintenance. During a shutdown, the pump(s) driving the oil through the pipeline are stopped causing the flow of oil within the pipeline to stop. The temperature of oil sitting within the pipeline during a shutdown will typically decrease to the ambient temperature. Therefore, pipelines operated in cold environments are particularly susceptible to gelation during a shutdown of the pipeline.

Pipeline gelation can be a substantial disruption to the operation of the pipeline, particularly for a large energy company that operates hundreds or thousands of miles of pipeline. Existing approaches to analyzing gelation are overly simplistic and do not account for large scale phenomena such as shearing of wax before a shutdown occurs. In one aspect, existing approaches involve determining gel yielding characteristics and calculating the pressure gradient required to break gels formed within a pipeline in order to restart flow. However, these approaches are reactive as opposed to preventative in that they do not provide a solution that avoids the occurrence of pipeline gelation in the first place.

With respect to existing approaches for preventing gelation, given the limitations with known techniques for analyzing pipeline gelation, the existing approaches to managing pipeline gelation risk rely on overdesign and overly conservative estimates such as limiting the amount of waxy crude oil, applying heat or insulation to the pipeline, or using additives to reduce the gelation temperature point. In some cases, waxy crude must be transported by means that are substantially less efficient than a pipeline, such as truck or rail. Relying on overdesign and overly conservative estimates to avoid the risk of pipeline gelation results in inefficiency and unnecessary limitations in the operation of pipeline networks. Therefore, a more sophisticated approach that more accurately monitors and predicts pipeline gelation would be beneficial.

### SUMMARY

The present disclosure is directed to monitoring and predicting gelation risk in a pipeline. In one example embodiment, the present disclosure is directed to a computing system comprising a processor, a memory, and a storage device comprising a gelation analytics tool. The gelation analytics tool can comprise computer-executable instructions that: a) generate a wax precipitation model of a percentage of wax precipitation over a temperature range for a batch of crude oil, the wax precipitation model based upon thermodynamic behavior of wax in the batch of crude oil; b) generate a pipeline temperature model of predicted temperatures along a pipeline over a selected time period, the pipeline temperature model based upon forecasted ambient conditions about the pipeline during the selected time period; c) receive a proposed schedule comprising at least one shutdown of the pipeline during the selected time period for a new batch of crude oil; d) generate a wax precipitation prediction for the new batch of crude oil during the selected time period by applying the pipeline temperature model during the selected time period to the wax precipitation model; and e) output the wax precipitation prediction for managing the new batch of crude oil in the pipeline.

In another example embodiment, the present disclosure is directed to a method for managing gelation risk in a pipeline. The method can comprise: a) providing, by a gelation analytics tool, a wax precipitation model of a percentage of wax precipitation over a temperature range for a batch of crude oil, the wax precipitation model based upon thermodynamic behavior of wax in the batch of crude oil; b) providing, by the gelation analytics tool, a pipeline temperature model of predicted temperatures along the pipeline over a selected time period, the pipeline temperature model based upon forecasted ambient conditions about the pipeline during the selected time period; c) receiving a proposed schedule comprising at least one shutdown of the pipeline during the selected time period for a new batch of crude oil; d) generating, by the gelation analytics tool, a wax precipitation prediction for the new batch of crude oil during the selected time period by applying the pipeline temperature model during the selected time period to the wax precipitation model; and e) outputting, by the gelation analytics tool, the wax precipitation prediction for managing the new batch of crude oil in the pipeline.

In yet another example embodiment, the present disclosure is directed to a computer-readable medium comprising instructions that when executed by a processor perform a method. The method can comprise: a) providing, by a gelation analytics tool, a wax precipitation model of a percentage of wax precipitation over a temperature range for a batch of crude oil, the wax precipitation model based upon thermodynamic behavior of wax in the batch of crude oil; b) providing, by the gelation analytics tool, a pipeline temperature model of predicted temperatures along the pipeline over a selected time period, the pipeline temperature model based upon forecasted ambient conditions about the pipeline during the selected time period; c) receiving a proposed schedule comprising at least one shutdown of the pipeline during the selected time period for a new batch of crude oil; d) generating, by the gelation analytics tool, a wax precipitation prediction for the new batch of crude oil during the selected time period by applying the pipeline temperature model during the selected time period to the wax precipitation model; and e) outputting, by the gelation analytics tool, the wax precipitation prediction for managing the new batch of crude oil in the pipeline.

The foregoing embodiments are non-limiting examples and other aspects and embodiments will be described herein. The foregoing summary is provided to introduce various concepts in a simplified form that are further described below in the detailed description. This summary is not intended to identify required or essential features of the claimed subject matter nor is the summary intended to limit the scope of the claimed subject matter.

### BRIEF DESCRIPTION OF THE DRAWINGS

The accompanying drawings illustrate only example embodiments of a system, method, and computer-readable media for monitoring and predicting gelation risk in a pipeline. Therefore, the examples provided are not to be considered limiting of the scope of this disclosure. The principles illustrated in the example embodiments of the drawings can be applied to alternate methods and apparatus. Additionally, the elements and features shown in the drawings are not necessarily to scale, emphasis instead being placed upon clearly illustrating the principles of the example embodiments. Certain dimensions or positions may be exaggerated to help visually convey such principles. In the drawings, the same reference numerals used in different embodiments designate like or corresponding, but not necessarily identical, elements.

FIG. 1 illustrates an example of a pipeline and a computing system that monitors the risk of gelation in the pipeline in accordance with an example embodiment of the disclosure.

FIG. 2A illustrates an example of wax precipitation models as a function of temperature in accordance with an example embodiment of the disclosure.

FIG. 2B illustrates an example of wax precipitation models as a function of temperature and pressure in accordance with an example embodiment of the disclosure.

FIG. 3 illustrates an example soil temperature sub-model along a pipeline in accordance with an example embodiment of the disclosure.

FIG. 4 illustrates an example soil moisture content sub-model along a pipeline in accordance with an example embodiment of the disclosure.

FIG. 5 illustrates an example pipeline temperature model along a length of a pipeline in accordance with an example embodiment of the disclosure.

FIG. 6 illustrates tracking of different blends of oil along a pipeline in accordance with an example embodiment of the disclosure.

FIG. 7 illustrates flow rate in a pipeline as a function of time in accordance with an example embodiment of the disclosure.

FIG. 8 illustrates a wax precipitation prediction for a batch of oil over time that is generated by the gelation analytics tool in accordance with an example embodiment of the disclosure.

FIGS. 9A and 9B are a flowchart illustrating an example method for calculating a wax precipitation prediction for a batch of oil over time using the gelation analytics tool in accordance with an example embodiment of the disclosure.

### DETAILED DESCRIPTION OF EXAMPLE EMBODIMENTS

The example embodiments discussed herein are directed to systems, methods, and computer-readable media for monitoring and predicting gelation risk in a pipeline. Unlike prior approaches that identify gel yielding characteristics and attempt to predict pressure gradient requirements to break a gel formed within a pipeline, the solutions described herein seek to predict and avoid pipeline gelation based on the current conditions of the pipeline and the characteristics of the batches of crude oil flowing through the pipeline. The example embodiments of the present disclosure are an improvement over prior approaches because they use a gelation analytics tool that relies upon thermodynamic models and/or empirical data to predict when gelation is likely to occur in a pipeline. Using empirical data and/or thermodynamic models provides a leading indicator that the pipeline operator can use to monitor and control gelation risk in a pipeline. The data analyzed by the gelation analytics tool can include temperature and pressure data for blends of crude oil containing varying amounts of wax components to generate a wax precipitation model. The gelation analytics tool also can utilize or analyze weather and environmental conditions for the pipeline to generate a model of temperature along the pipeline.

By performing a robust analysis of the temperature conditions along the pipeline and the properties of the crude oil in the pipeline, the gelation analytics tool described herein can support monitoring of pipelines and predicting when gelation is a risk. Being able to predict gelation risk with accuracy assists a pipeline operator in managing the operation of the pipeline and when the pipeline can be shutdown. The gelation analytics tool also can be used in decisions about blending different batches of crude oil having different wax content in order to maximize throughput of waxy crude oil in the pipeline. Considering the costs and downtime associated with pipeline gelation, the ability to more accurately predict pipeline gelation substantially improves the operation of pipelines. Additionally, more accurate monitoring for pipeline gelation improves efficiency in that prior approaches that involve overdesign or overly conservative estimates can be eliminated. As will be described further in the following examples, the systems, methods, and computer-readable media described herein improve upon existing approaches to managing the risk of pipeline gelation. The improved approaches described herein can be embodied in a computer system, as a method executed by a computer system, and as a method embodied in a computer-readable medium.

In the following paragraphs, particular embodiments will be described in further detail by way of example with reference to the drawings. In the description, well-known components, methods, and/or processing techniques are omitted or briefly described. Furthermore, reference to various feature(s) of the embodiments is not to suggest that all embodiments must include the referenced feature(s).

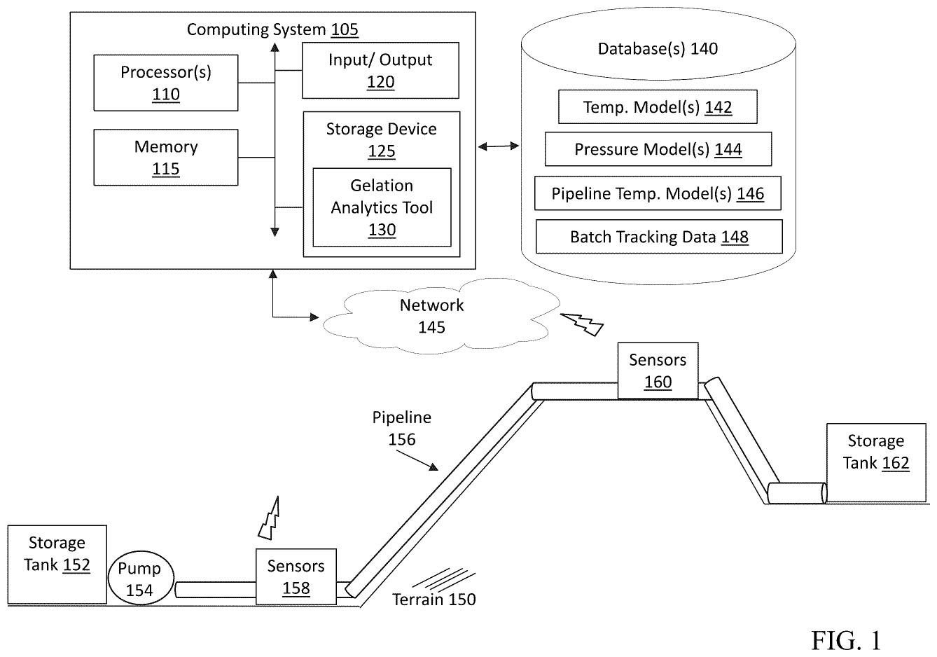

Referring now to FIG. 1, an example system for managing gelation risk in a pipeline is illustrated. As explained previously, pipelines that transport crude oil can be found throughout the world in a variety of environments, including onshore and offshore (undersea) environments. For illustrative purposes, FIG. 1 provides a simplified illustration of a crude oil pipeline in an onshore environment that traverses a terrain having varying elevation. It should be understood that the system and the pipeline illustrated in FIG. 1 provide simplified representations and, in reality, pipelines and the systems used to manage pipelines can include a substantial number of complex subsystems and components.

As illustrated in FIG. 1, crude oil can be stored in a storage tank 152 and can be pumped by a pump 154 through a pipeline 156. While a single storage tank 152 is illustrated at the beginning of pipeline 156 it should be understood that the pipeline can receive batches of crude oil from various sources, including other storage tanks and pipelines. The pipeline 156 can be hundreds of miles long and traverses terrain 150 which has varying elevations. The varying elevations of the terrain 150 can affect the ambient environmental conditions surrounding the pipeline 156, including air and soil temperatures, air and soil moisture levels, air pressure, and precipitation. Furthermore, portions of the pipeline may be subterranean which can further impact the crude oil flowing through the pipeline. In the example of FIG. 1, the pipeline 156 transports crude oil over a mountain to a second storage tank 162. However, the techniques described herein are applicable to pipelines that can be found in a wide variety of environments.

The system of FIG. 1 also illustrates a computing system 105 and a database 140 that are used to manage the crude oil flowing through the pipeline and prevent pipeline gelation. The computing system 105 can be located in proximity to the pipeline or can be distributed remotely as a cloud computing service. The computing system 105 can receive data from sensors 158 and 160 positioned along the pipeline. The sensors 158 and 160 can include temperature sensors that can measure temperatures external or internal to the pipeline, pressure sensors that can measure pressures external or internal to the pipeline, and moisture sensors that can measure moisture levels along the exterior of the pipeline. The sensors 158 and 160 include wireless transmitters that communicate the data collected by the sensors via network 145 to the computing system 105. In addition to the data from sensors 158 and 160, or as an alternative, the computing system 105 can receive data from other sources including monitoring equipment at the beginning and end of the pipeline 156 as well as environmental data from weather observation systems or other data logging systems.

As is commonly known for computing systems, computing system 105 includes one or more processors 110, memory 115, and input/output interfaces 120. A storage device 125 can be an integral component of the computing system 105, as illustrated in FIG. 1, or it can be external to the computing system. In addition to an operating system, the storage device can include a gelation analytics tool 130 in accordance with the embodiments described herein. The gelation analytics tool 130 can receive a variety of data via network 145 and can analyze the data to provide wax precipitation predictions for the purpose of avoiding pipeline gelation. The gelation analytics tool includes computer executable instructions that perform methods for analyzing data associated with the crude oil flowing through the pipeline and predicting the amount of wax precipitation. The data the gelation analytics tool receives can include the previously described sensor data and environmental data, as well as data models that describe the thermodynamic characteristics of batches of crude oil and the environmental conditions surrounding the pipeline.

A variety of data models can be stored in the database 140. As will be described further below, the data models can include temperature models 142, pressure models 144, and pipeline temperature models 146. The temperature models 142 and pressure models 144 describe the behavior of batches of crude oil indicating the percentage of wax precipitation in the crude oil as temperature and pressure change. The percentage of wax precipitation as determined by the temperature and pressure models provides an indication of when a risk of gelation may occur with the batch of crude oil. Separate temperature models can be created for different batches of crude oil depending upon the amount of waxy crude oil present in the batch. Similarly, separate pressure models can be created for different batches of crude oil depending upon the amount of waxy crude oil present in the batch. Lastly, the database also can store batch tracking data 148 indicating the position of various batches in the pipeline. Batch tracking data 148 is useful because different batches of crude oil can have different amounts of wax that affect the likelihood of gelation. The batch tracking data 148 also is useful because varying thermodynamic and environmental conditions may be present at different locations along the pipeline, such as a section of pipeline traversing a mountain as compared to a section of pipeline at a lower elevation.

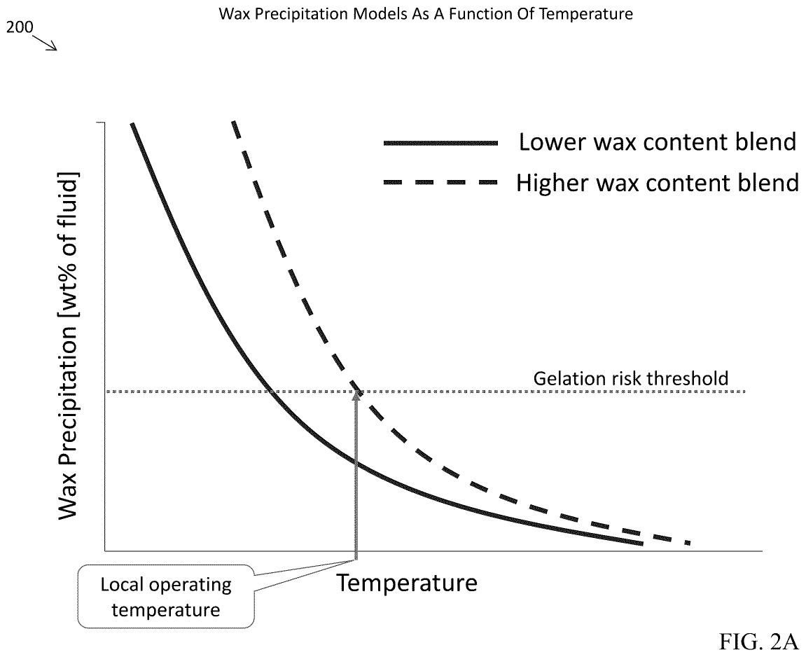

It should be understood that the computing components illustrated in FIG. 1 are merely illustrative examples and that in alternate embodiments certain of the computing components can be combined, simplified, or distributed in a different manner. While the gelation analytics tool 130 is illustrated in FIG. 1 as a software module stored in storage device 125 of computing system 105, it should be understood that the gelation analytics tool 130 also can be implemented as services available on remote computing devices, such as a cloud computing environment, which services can be accessed to analyze thermodynamic data and models to provide wax precipitation predictions. Additionally, while the gelation analytics tool 130 is described herein as a single software-implemented tool, in other embodiments the gelation analytics tool can be distributed among multiple software modules or services. In certain embodiments, the gelation analytics tool 130 can include a machine learning model that is trained from historical data relating to thermodynamic conditions and crude oil characteristics to more accurately predict wax precipitation in pipelines. The operation of the gelation analytics tool 130 will be described in further detail below in connection with the example models and methods of FIGS. 2A through 9B.

Referring to FIG. 2A, examples of wax precipitation models as a function of temperature are illustrated for two different batches of crude oil. The wax precipitation models illustrated in FIG. 2A can be used to predict the risk of gelation in the pipeline. The first batch is a blend of crude oils containing a lower wax content and the wax precipitation model for this first batch is illustrated by the solid line curve in the graph. The second batch is a blend of crude oils containing a higher wax content than the first batch and the wax precipitation model for this second batch is illustrated by the dotted line curve in the graph. As illustrated in the graph, both the first wax precipitation model and the second wax precipitation model indicate that the amount of wax precipitation in both blends of crude oil will increase as the temperature decreases, with the rate of wax precipitation being greater in the second model corresponding to the batch with a greater amount of wax. As an example, the graph in FIG. 2A also notes a local operating temperature at a selected point along the pipeline. The local operating temperature can be calculated by a dynamic hydraulic flow simulation of the pipeline. Alternatively or in addition to the simulation, local measurements of conditions along the pipeline can be received from the sensors located along the pipeline. At the local operating temperature, the second wax precipitation model indicates the second batch of crude oil with the higher wax content will approach, but not exceed a gelation risk threshold. The gelation risk threshold is the level of wax precipitation where the batch of crude oil is likely to solidify into a gel. Accordingly, the wax precipitation models allow the pipeline operator to plan to pump the second batch of crude oil containing the higher wax content at the local operating temperature without risking pipeline gelation. The ability to pump the second batch of crude oil through the pipeline, as opposed to less efficient transport methods such as truck or rail, provides a significant advantage.

The wax precipitation models can be generated using thermodynamic simulations of the behavior of crude oil at various temperatures and pressures. Alternatively or in addition to the thermodynamic simulations, the wax precipitation models can be developed from empirical data gather during testing and observation of the crude oil. Once the wax precipitation models are generated, they also can facilitate more complex planning such as the blending of different batches of crude oil. As one example, in connection with planning for the pumping of a crude oil batch through the pipeline, if the pipeline operator estimates a second local operating temperature that is lower than the local operating temperature indicated in FIG. 2A, a crude oil batch having wax content matching that of the second wax precipitation model will exceed the gelation risk threshold and be likely to solidify in the pipeline causing a shutdown. The pipeline operator can avoid this potential gelation problem by blending two batches of crude oil having higher and lower wax content in order to bring the blended batch below the gelation risk threshold at the second local operating temperature. A new wax precipitation model can be generated for the blended batch by combining the wax precipitation models of the two underlying batches of oil that are blended. Thus, the wax precipitation models allow for more sophisticated blending of waxy crude oil batches to maximize the throughput of waxy crude oil through the pipeline.

FIG. 2B is similar to FIG. 2A, but illustrates two examples of wax precipitation models as a function of temperature and pressure for two different batches of crude oil. In systems where pipeline pressures vary over a large range, such as a range from near ambient pressure to several orders of magnitude greater than ambient pressure, wax precipitation characteristics can vary significantly. When the variation in wax precipitation is a function of both pressure and temperature, wax precipitation models can be constructed to capture both dependencies. The wax precipitation models illustrated in FIG. 2B can be used to predict the risk of gelation in the pipeline based upon different temperatures and pressures within the pipeline. The first batch is a blend of crude oils containing a lower wax content and the wax precipitation model for this first batch is illustrated by the solid line curve in the graph. The second batch is a blend of crude oils containing a higher wax content than the first batch and the wax precipitation model for this second batch is illustrated by the dotted line curve in the graph. As illustrated in the graph, both the first wax precipitation model and the second wax precipitation model indicate that the amount of wax precipitation in both blends of crude oil will increase as the temperature decreases, with the rate of wax precipitation being greater in the second model corresponding to the batch with a greater amount of wax. As an example, the graph in FIG. 2B also notes a local operating temperature and pressure at a selected point along the pipeline. The local operating temperature and pressure can be calculated by a dynamic hydraulic flow simulation of the pipeline. Alternatively, the local operating temperature and pressure within the pipeline can be measured with local sensors along the pipeline. The use of wax precipitation models as a function of temperature and pressure can be particularly useful during a shutdown of the pipeline when pressure within the well can drop substantially.

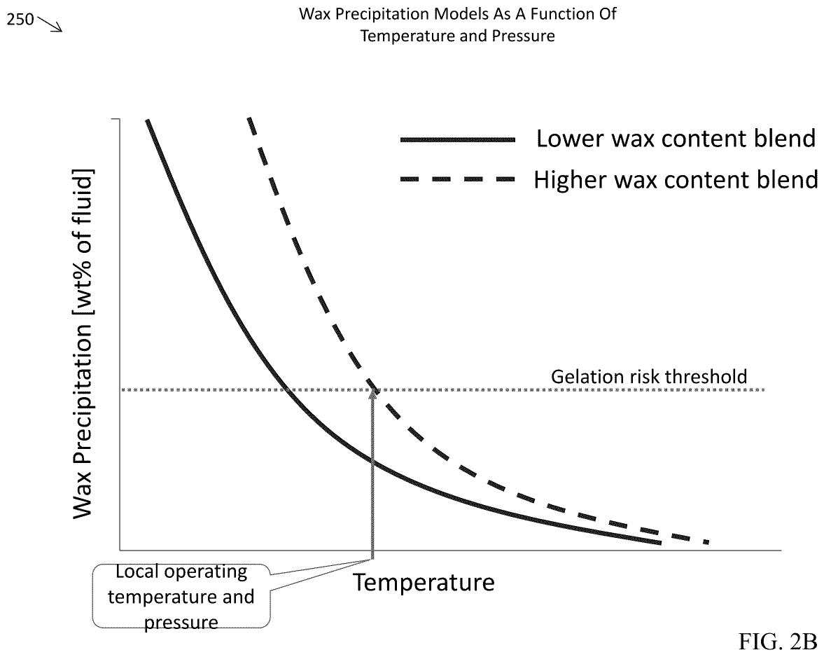

Referring now to FIGS. 3, 4, and 5, the generation of a pipeline temperature model will be described. One or more pipeline temperature models can be used to generate estimates of local operating temperatures along the pipeline which the gelation analytics tool uses in combination with wax precipitation models to predict the risk of gelation in the pipeline. Because a variety of environmental factors affect the temperature along the pipeline, the pipeline temperature modeling allows the gelation analytics tool to more accurately predict the risk of gelation.

As the crude oil flows through the pipeline, it loses heat to the pipeline and the air and soil surrounding the pipeline. The calculation of heat lost from the flowing crude oil is critical to estimating the current gelation risk and to projecting future gelation risk in the pipeline. As explained previously, the risk of pipeline gelation is particularly high when the pumps are stopped and the pipeline is shutdown, such as for maintenance. Therefore, generating temperature models that describe the pipeline both during flowing and non-flowing states allows for more accurate predictions of gelation.

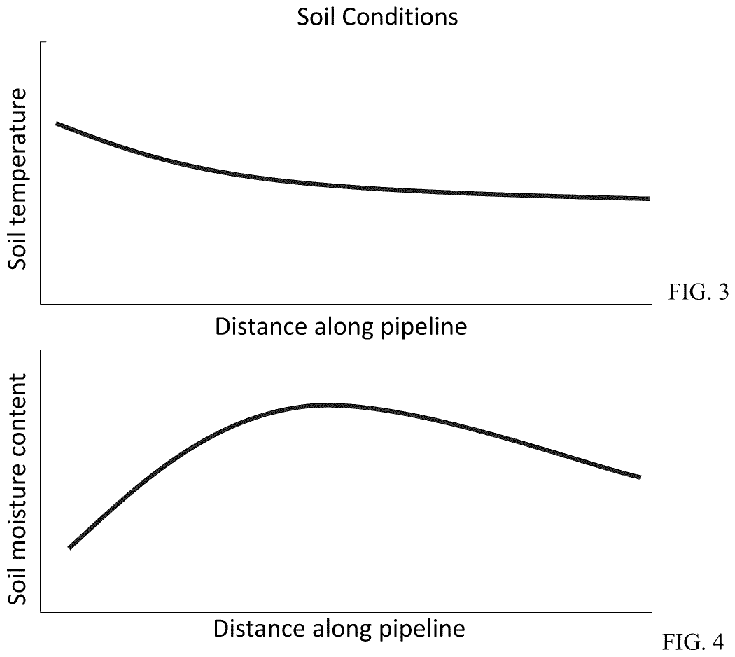

As illustrated in FIGS. 3 and 4, local soil temperature and local soil water content along the pipeline are used in calculating heat transfer from the crude oil in the pipeline. Soil temperature and soil moisture content vary throughout the year and also can vary along the pipeline, particularly when comparing mountainous regions and regions of lower elevation. Accordingly, soil temperatures and soil water content are inputs to the gelation analytics tool for generating a pipeline temperature model. The gelation analytics tool can receive these inputs from sensors located along the pipeline as illustrated in FIG. 1. Alternatively or in addition, the gelation analytics tool can obtain weather data for incorporating into the pipeline temperature model. Furthermore, in other embodiments, such as for an undersea pipeline, environmental data relating to the temperature of the water can be incorporated into the pipeline temperature model.

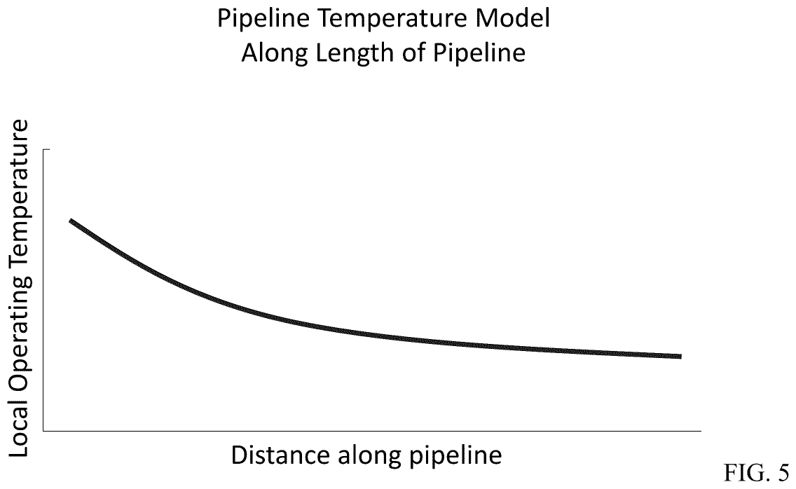

FIG. 5 shows one example of a pipeline temperature model illustrated as a curve in the graph. In the example of FIG. 5, the pipeline temperature model indicates that the local operating temperature along the pipeline will decrease as distance along the pipeline increases. The gelation analytics tool can generate several pipeline temperature models to address various situations. For example, a current pipeline temperature model can describe the current temperatures along the pipeline based on the recent sensor data and/or weather data provided to the gelation analytics tool. The current pipeline temperature model can be used in conducting real-time analyses of gelation risk in the pipeline. As another example, a predicted pipeline temperature model can describe the predicted temperatures along the pipeline at a selected time, such as the time of a scheduled shutdown of the pipeline. Accordingly, the gelation analytics tool can use a pipeline temperature model that most accurately corresponds with the time period of interest for assessing gelation risk in the pipeline.

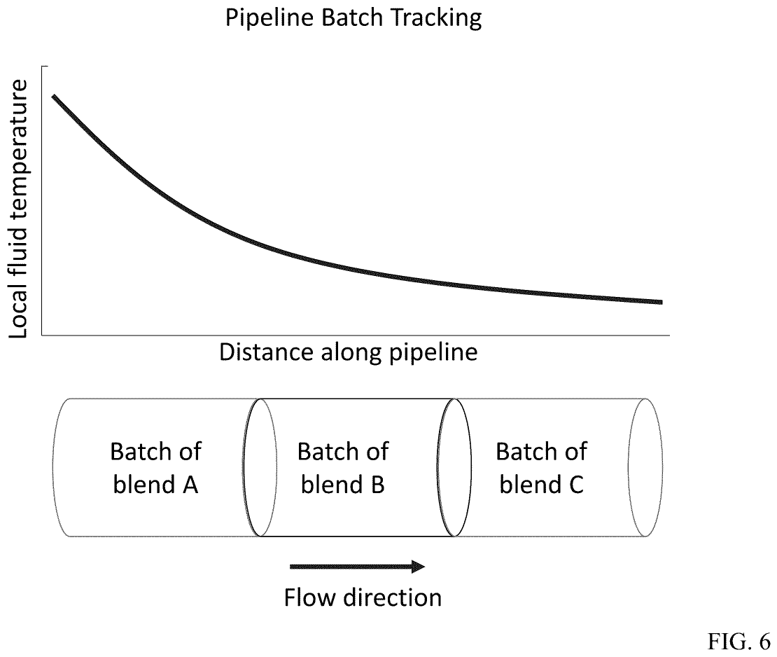

Referring now to FIG. 6, the gelation analytics tool also can use batch tracking data in its analysis. As explained previously, different batches of crude oil often flow through a pipeline and those different batches can have varying levels of gelation risk depending upon the amount of wax in the batch. Data describing the position and temperature of each crude oil batch can be stored in a database and analyzed by the gelation analytics tool to more accurately assess gelation risk for each batch. As illustrated in FIG. 6, a pipeline can have three different crude oil batches (batch A, batch B, and batch C) flowing through the pipeline at a flow rate. Each of batches A, B, and C can have unique blends of waxy crudes and unique wax precipitation behavior as a function of temperature and pressure. A unique wax precipitation model can be applied to each batch corresponding to the wax characteristics of each particular batch. The pipeline temperature models described previously can be used to dynamically track the local operating temperatures and the change in those temperatures as the batches move through the pipeline. Accurately matching the local operating temperature for each batch allows the gelation analytics tool to more accurately assess the gelation risk.

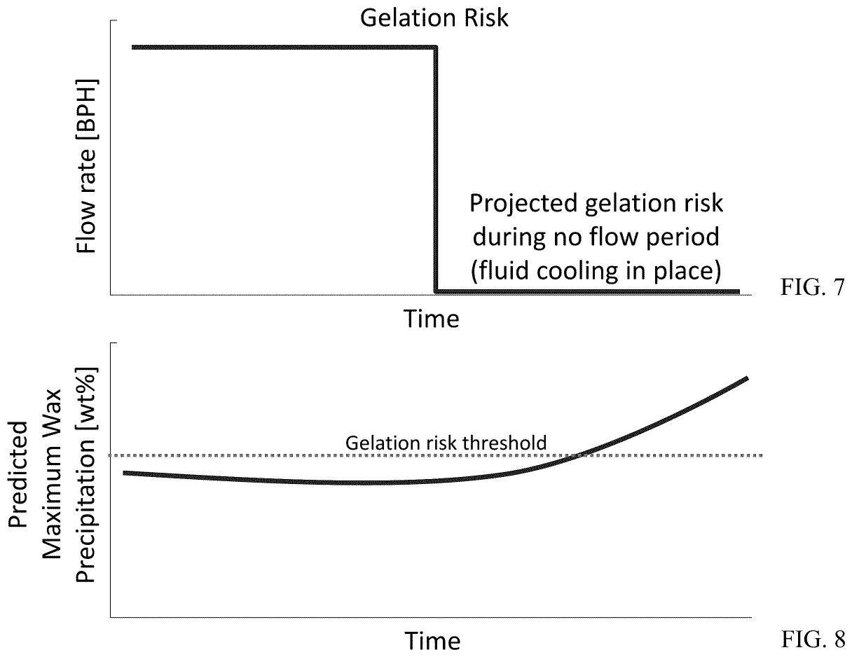

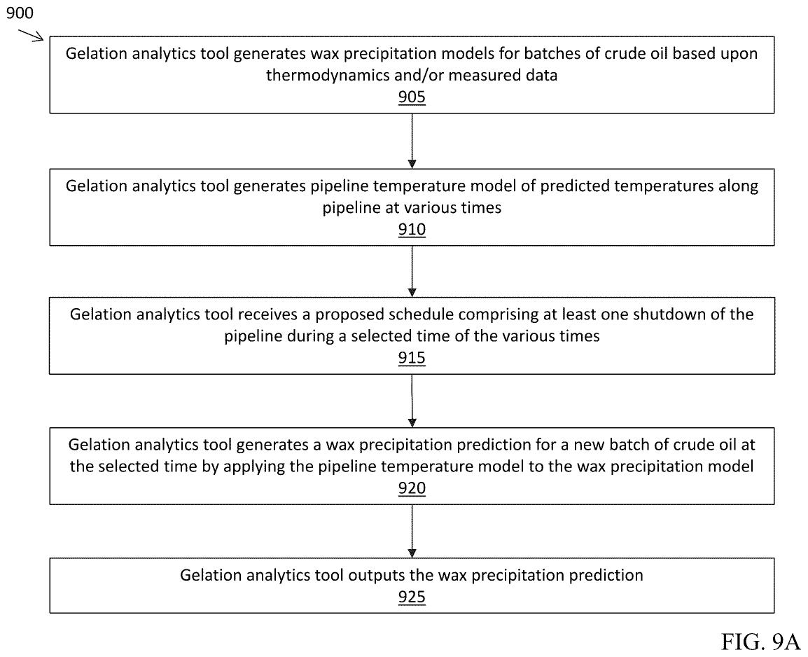

Referring now to FIGS. 7 and 8, two graphs are provided to illustrate how the gelation analytics tool monitors gelation risk. FIG. 7 illustrates flow rate through a pipeline. The drop in flow rate can be associated with a scheduled shutdown of the pipeline for maintenance. It is during this scheduled shutdown that there is a higher risk of gelation because the crude oil loses heat to the surrounding environment while it is relatively stationary within the pipeline. The graph in FIG. 8 illustrates a prediction of wax precipitation generated by the gelation analytics tool by applying a pipeline temperature model to a wax precipitation model for the crude oil batch that is passing through the pipeline at the time of the scheduled shutdown. Where there are multiple different crude oil batches in the pipeline during the planned shutdown, the operator may choose to focus on the highest risk batch containing the most wax. As the graph in FIG. 8 shows, the gelation analytics tool predicts increasing wax precipitation while the crude oil sits in the pipeline during the scheduled shutdown. At a certain point during the scheduled shutdown, the gelation analytics tool predicts the percentage of wax precipitation in the pipeline will exceed a predetermined gelation risk threshold. This prediction from the gelation analytics tool allows the pipeline operator to make adjustments to reduce the risk of gelation. As one example, the planned shutdown of the pipeline can be rescheduled to another time when the environmental temperatures will be warmer resulting in less wax precipitation. As another example, the pipeline operator may choose to blend the waxy crude oil with one or more other crude oil batches containing less wax, thereby reducing the wax precipitation during the planned shutdown.

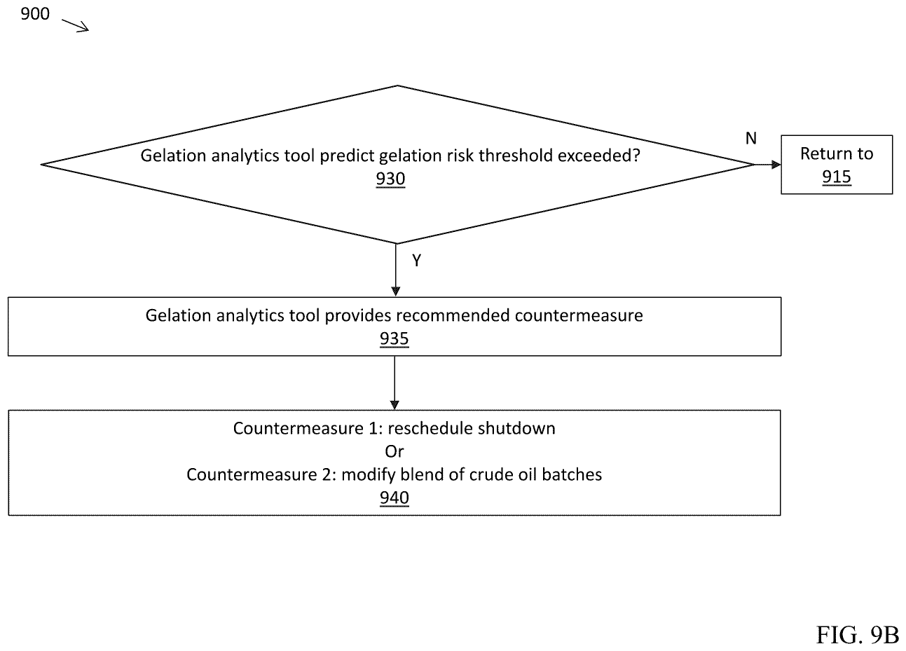

Referring now to FIGS. 9A and 9B, an example method 900 is illustrated for using the gelation analytics tool to analyze wax precipitation models and pipeline temperature models to predict the risk of gelation in a pipeline. In alternate example embodiments, one or more of the operations illustrated in FIGS. 9A and 9B could be performed in parallel, performed a different sequence, or eliminated. Furthermore, in alternate embodiments, other operations may be added to the method of FIGS. 9A and 9B.

Beginning with operation 905, the gelation analytics tool generates wax precipitation models for batches of crude oil. As described previously, the wax precipitation models can be generated from one or both of thermodynamic characteristics of the crude oil and data collected from observations of the behavior of the crude oil in response to pressure and temperature changes. Unique wax precipitation models can be created that correspond to the wax content of unique crude oil batches. The gelation analytics tool can store the wax precipitation models in a database for later use.

In operation 910, the gelation analytics tool generates one or more pipeline temperature models of predicted temperatures along the pipeline at various times. As one example, a current pipeline temperature model can provide a real-time description of the different temperatures currently present along the pipeline. As another example, a predicted pipeline temperature model can provide an estimate of temperatures along the pipeline at a future time. The gelation analytics tool generates the pipeline temperature models from data it receives from various sources. The sources of data can include thermodynamic models of conditions along the pipeline, environmental data collected from sensors along the pipeline, and current or forecasted weather data. The gelation analytics tool stores the pipeline temperature models in a database for subsequent use in evaluating the risk of gelation.

In operation 915, the gelation analytics tool receives a proposed schedule comprising at least one shutdown of the pipeline during a selected time. Because a shutdown of the pipeline can present a higher risk of pipeline gelation, the pipeline operator can use the gelation analytics tool to assess the risk during the shutdown.

In operation 920, the gelation analytics tool can retrieve a pipeline temperature model for the time of the scheduled shutdown. For example, if the shutdown is scheduled for the daytime hours during the month of December, the gelation analytics tool can retrieve a predicted pipeline temperature model that predicts temperatures along the pipeline during the daytime in December. The gelation analytics tool also retrieves a wax precipitation model that accurately describes the behavior of the batch of crude oil that will be present in the pipeline during the scheduled shutdown. If multiple batches will be in the pipeline during the scheduled shutdown, the gelation analytics tool can select the wax precipitation model corresponding to the crude oil batch containing the highest amount of wax as that is the batch that will present the greatest risk of gelation. The gelation analytics tool applies the predicted pipeline temperature model to the selected wax precipitation model to predict the amount of wax precipitation during the scheduled shutdown. For example, the gelation analytics tool can apply the temperatures along those portions of the pipeline that are predicted to be the lowest in the pipeline temperature model to the wax precipitation model to predict the amount of wax precipitation. A gelation risk threshold can be set as a predetermined wax precipitation percentage in the wax precipitation model.

In operation 925, the gelation analytics tool can output a report of the wax precipitation prediction. The report can be one or more graphs illustrating the predicted wax precipitation at the time of the scheduled shutdown for various points along the pipeline. The gelation analytics tool also can be configured to generate an alert if a risk of gelation is predicted.

In operation 930, the pipeline operator decides whether any actions are needed in response to the wax precipitation prediction from the gelation analytics tool. If the gelation analytics tool indicates there is no risk of exceeding the gelation threshold, method 900 can return to operation 915 where the tool can be used for additional assessments of current or future conditions in the pipeline. Alternatively, if the gelation analytics tool predicts that the gelation risk threshold will be exceeded, the tool can provide recommended countermeasures in operation 935. As indicated in operation 940, one type of countermeasure would be to recommend that the scheduled shutdown be rescheduled for another time when less waxy crude is flowing through the pipeline or when the pipeline temperature model indicates warmer temperatures along the pipeline. Another type of countermeasure is to modify the blend of crude oil that will be flowing through the pipeline during the scheduled shutdown so that the blend has a lower wax content. While the example method 900 describes using the gelation analytics tool to plan for a scheduled shutdown, the method can be modified for other situations. For example, the gelation analytics tool can be operated periodically to provide current assessments of gelation risk in the pipeline by using a pipeline temperature model and wax precipitation model that correspond to the current conditions along the pipeline.

### General Information Regarding Computing Systems

As described in connection with FIGS. 1-9B, some or all of the processing operations described in connection with the foregoing systems and methods can be performed by computing systems such as a personal computer, a desktop computer, a computer server, or cloud computing systems. As explained previously, certain operations of the foregoing methods can be performed by a combination of computing systems.

The computing systems used in the foregoing embodiments can include typical components such as one or more processors, memories, input/output devices, and a storage devices. The components of the computing systems can be interconnected, for example, by a system bus or by communication links. The components of the previously described computing systems are not exhaustive.

The one or more processors can be one or more hardware processors and can execute computer-readable instructions, such as instructions stored in a memory. The processor can be an integrated circuit, a central processing unit, a multi-core processing chip, an SoC, a multi-chip module including multiple multi-core processing chips, or other hardware processor in one or more example embodiments. The hardware processor is known by other names, including but not limited to a computer processor, a microprocessor, and a multi-core processor.

The memory can store information including computer-readable instructions and data. The memory can be cache memory, a main memory, and/or any other suitable type of memory. The memory is a non-transitory computer-readable medium. In some cases, the memory can be a volatile memory device, while in other cases the memory can be a non-volatile memory device.

The storage device can be a non-transitory computer-readable medium that provides large capacity storage for a computing system. The storage device can be a disk drive, a flash drive, a solid state device, or some other type of storage device. In some cases, the storage device can be a database that is remote from the computing system. The storage device can store operating system data, file data, database data, algorithms, and software modules, as examples.

### Example Embodiments

The following clauses provide illustrative example embodiments.

[EE1] A computing system comprising:
a processor;
a memory; and
a storage device comprising a gelation analytics tool, the gelation analytics tool comprising computer-executable instructions that:
   generate a wax precipitation model of a percentage of wax precipitation over a temperature range for a batch of crude oil, the wax precipitation model based upon thermodynamic behavior of wax in the batch of crude oil;
   generate a pipeline temperature model of predicted temperatures along a pipeline over a selected time period, the pipeline temperature model based upon forecasted ambient conditions about the pipeline during the selected time period;
   receive a proposed schedule comprising at least one shutdown of the pipeline during the selected time period for a new batch of crude oil;
   generate a wax precipitation prediction for the new batch of crude oil during the selected time period by applying the pipeline temperature model during the selected time period to the wax precipitation model; and
   output the wax precipitation prediction for managing the new batch of crude oil in the pipeline.

[EE2] The computing system of EE1, wherein the wax precipitation model is based upon measurements of wax precipitation in the batch of crude oil.

[EE3] The computing system of EE1, wherein the wax precipitation model includes a gelation risk threshold for an identified value of the percentage of wax precipitation.

[EE4] The computing system of EE3, wherein when the wax precipitation prediction indicates the gelation risk threshold will be exceeded during the selected time period for the new batch of crude oil, the gelation analytics tool recommends the at least one shutdown is modified to reduce a likelihood of gelation occurring for the new batch of crude oil in the pipeline.

[EE5] The computing system of EE1, wherein the gelation analytics tool determines a position of the new batch of crude oil in the pipeline based upon a flow rate of the new batch of crude oil.

[EE6] The computing system of EE1, wherein the new batch of crude oil is a mixture of a first batch of crude oil and a second batch of crude oil and wherein the gelation analytics tool uses flow rate data to determine a proportion of the first batch of crude oil and a proportion of the second batch of crude oil in the new batch of crude oil.

[EE7] The computing system of EE6, wherein the wax precipitation model comprises a first wax precipitation model and a second wax precipitation model.

[EE8] The computing system of EE6,
wherein the first batch of crude oil has a first wax component comparable to data upon which the first wax precipitation model is based; and
wherein the second batch of crude oil has a second wax component comparable to data upon which the second wax precipitation model is based.

[EE9] The computing system of EE1, wherein the pipeline is located in soil and the forecasted ambient conditions include forecasted weather and forecasted soil conditions.

[EE10] The computing system of EE1, wherein the pipeline is located above ground and the forecasted ambient conditions include forecasted weather.

[EE11] The computing system of EE1, wherein the pipeline is located undersea and the forecasted ambient conditions include forecasted water temperatures.

[EE12] A computer-implemented method for managing gelation risk in a pipeline, the method comprising:
providing, by a gelation analytics tool, a wax precipitation model of a percentage of wax precipitation over a temperature range for a batch of crude oil, the wax precipitation model based upon thermodynamic behavior of wax in the batch of crude oil;
providing, by the gelation analytics tool, a pipeline temperature model of predicted temperatures along the pipeline over a selected time period, the pipeline temperature model based upon forecasted ambient conditions about the pipeline during the selected time period;
receiving a proposed schedule comprising at least one shutdown of the pipeline during the selected time period for a new batch of crude oil;
generating, by the gelation analytics tool, a wax precipitation prediction for the new batch of crude oil during the selected time period by applying the pipeline temperature model during the selected time period to the wax precipitation model; and
outputting, by the gelation analytics tool, the wax precipitation prediction for managing the new batch of crude oil in the pipeline.

[EE13] The computer-implemented method of EE12, wherein when the wax precipitation prediction indicates a gelation risk threshold will be exceeded during the selected time period for the new batch of crude oil, the gelation analytics tool recommends the at least one shutdown is modified to reduce a likelihood of gelation occurring for the new batch of crude oil in the pipeline.

[EE14] The computer-implemented method of EE12, wherein the gelation analytics tool determines a position of the new batch of crude oil in the pipeline based upon a flow rate of the new batch of crude oil.

[EE15] The computer-implemented method of EE12, wherein the new batch of crude oil is a mixture of a first batch of crude oil and a second batch of crude oil and wherein the gelation analytics tool uses flow rate data to determine a proportion of the first batch of crude oil and a proportion of the second batch of crude oil in the new batch of crude oil.

[EE16] The computer-implemented method of EE15,
wherein the first batch of crude oil has a first wax component comparable to data upon which the first wax precipitation model is based; and
wherein the second batch of crude oil has a second wax component comparable to data upon which the second wax precipitation model is based.

[EE17] The computer-implemented method of EE12, wherein the forecasted ambient conditions include one or more of forecasted weather, forecasted soil conditions, and forecasted water temperatures.

[EE18] A computer-readable medium comprising instructions that when executed by a processor perform a method comprising:
providing, by a gelation analytics tool, a wax precipitation model of a percentage of wax precipitation over a temperature range for a batch of crude oil, the wax precipitation model based upon thermodynamic behavior of wax in the batch of crude oil;
providing, by the gelation analytics tool, a pipeline temperature model of predicted temperatures along the pipeline over a selected time period, the pipeline temperature model based upon forecasted ambient conditions about the pipeline during the selected time period;
receiving a proposed schedule comprising at least one shutdown of the pipeline during the selected time period for a new batch of crude oil;
generating, by the gelation analytics tool, a wax precipitation prediction for the new batch of crude oil during the selected time period by applying the pipeline temperature model during the selected time period to the wax precipitation model; and
outputting, by the gelation analytics tool, the wax precipitation prediction for managing the new batch of crude oil in the pipeline.

[EE19] The computer-readable medium of EE18, wherein when the wax precipitation prediction indicates a gelation risk threshold will be exceeded during the selected time period for the new batch of crude oil, the gelation analytics tool recommends the at least one shutdown is modified to reduce a likelihood of gelation occurring for the new batch of crude oil in the pipeline.

[EE20] The computer-readable medium of EE18, wherein the new batch of crude oil is a mixture of a first batch of crude oil and a second batch of crude oil and wherein the gelation analytics tool uses flow rate data to determine a proportion of the first batch of crude oil and a proportion of the second batch of crude oil in the new batch of crude oil.

### Assumptions and Definitions

For any figure shown and described herein, one or more of the components may be omitted, added, repeated, and/or substituted. Accordingly, embodiments shown in a particular figure should not be considered limited to the specific arrangements of components shown in such figure. Further, if a component of a figure is described but not expressly shown or labeled in that figure, the label used for a corresponding component in another figure can be inferred to that component. Conversely, if a component in a figure is labeled but not described, the description for such component can be substantially the same as the description for the corresponding component in another figure.

With respect to the example methods described herein, it should be understood that in alternate embodiments, certain steps of the methods may be performed in a different order, may be performed in parallel, or may be omitted. Moreover, in alternate embodiments additional steps may be added to the example methods described herein. Accordingly, the example methods provided herein should be viewed as illustrative and not limiting of the disclosure.

The term “obtaining” may include receiving, retrieving, accessing, generating, etc. or any other manner of obtaining data.

Terms such as “first” and “second” are used merely to distinguish one element (or state of an element) from another. Such terms are not meant to denote a preference and are not meant to limit the embodiments described herein. In the example embodiments described herein, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

The terms “a,” “an,” and “the” are intended to include plural alternatives, e.g., at least one. The terms “including”, “with”, and “having”, as used herein, are defined as comprising (i.e., open language), unless specified otherwise.

Values, ranges, or features may be expressed herein as “about”, from “about” one particular value, and/or to “about” another particular value. When such values, or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In another aspect, use of the term “about” means ±20% of the stated value, ±15% of the stated value, ±10% of the stated value, ±5% of the stated value, ±3% of the stated value, or ±1% of the stated value.

Although embodiments described herein are made with reference to example embodiments, it should be appreciated by those skilled in the art that various modifications are well within the scope of this disclosure. Those skilled in the art will appreciate that the example embodiments described herein are not limited to any specifically discussed application and that the embodiments described herein are illustrative and not restrictive. From the description of the example embodiments, equivalents of the elements shown therein will suggest themselves to those skilled in the art, and ways of constructing other embodiments using the present disclosure will suggest themselves to practitioners of the art. Therefore, the scope of the example embodiments is not limited herein.

What is claimed is:

1. A computing system comprising:
a processor;
a memory; and
a storage device comprising a gelation analytics tool, the gelation analytics tool comprising computer-executable instructions that:
generate a wax precipitation model of a percentage of wax precipitation over a temperature range for a batch of crude oil, the wax precipitation model based upon thermodynamic behavior of wax in the batch of crude oil;
generate a pipeline temperature model of predicted temperatures along a pipeline over a selected time period, the pipeline temperature model based upon forecasted ambient conditions about the pipeline during the selected time period;
receive a proposed schedule comprising at least one shutdown of the pipeline during the selected time period for a new batch of crude oil;
generate a wax precipitation prediction for the new batch of crude oil during the selected time period by applying the pipeline temperature model during the selected time period to the wax precipitation model; and
output the wax precipitation prediction for managing the new batch of crude oil in the pipeline.

2. The computing system of claim 1, wherein the wax precipitation model is based upon measurements of wax precipitation in the batch of crude oil.

3. The computing system of claim 1, wherein the wax precipitation model includes a gelation risk threshold for an identified value of the percentage of wax precipitation.

4. The computing system of claim 3, wherein when the wax precipitation prediction indicates the gelation risk threshold will be exceeded during the selected time period for the new batch of crude oil, the gelation analytics tool recommends the at least one shutdown is modified to reduce a likelihood of gelation occurring for the new batch of crude oil in the pipeline.

5. The computing system of claim 1, wherein the gelation analytics tool determines a position of the new batch of crude oil in the pipeline based upon a flow rate of the new batch of crude oil.

6. The computing system of claim 1, wherein the new batch of crude oil is a mixture of a first batch of crude oil and a second batch of crude oil and wherein the gelation analytics tool uses flow rate data to determine a proportion of the first batch of crude oil and a proportion of the second batch of crude oil in the new batch of crude oil.

7. The computing system of claim 6, wherein the wax precipitation model comprises a first wax precipitation model and a second wax precipitation model.

8. The computing system of claim 6,
wherein the first batch of crude oil has a first wax component comparable to data upon which the first wax precipitation model is based; and
wherein the second batch of crude oil has a second wax component comparable to data upon which the second wax precipitation model is based.

9. The computing system of claim 1, wherein the pipeline is located in soil and the forecasted ambient conditions include forecasted weather and forecasted soil conditions.

10. The computing system of claim 1, wherein the pipeline is located above ground and the forecasted ambient conditions include forecasted weather.

11. The computing system of claim 1, wherein the pipeline is located undersea and the forecasted ambient conditions include forecasted water temperatures.

12. A computer-implemented method for managing gelation risk in a pipeline, the method comprising:
providing, by a gelation analytics tool, a wax precipitation model of a percentage of wax precipitation over a temperature range for a batch of crude oil, the wax precipitation model based upon thermodynamic behavior of wax in the batch of crude oil;
providing, by the gelation analytics tool, a pipeline temperature model of predicted temperatures along the pipeline over a selected time period, the pipeline temperature model based upon forecasted ambient conditions about the pipeline during the selected time period;
receiving a proposed schedule comprising at least one shutdown of the pipeline during the selected time period for a new batch of crude oil;
generating, by the gelation analytics tool, a wax precipitation prediction for the new batch of crude oil during the selected time period by applying the pipeline temperature model during the selected time period to the wax precipitation model; and
outputting, by the gelation analytics tool, the wax precipitation prediction for managing the new batch of crude oil in the pipeline.

13. The computer-implemented method of claim 12, wherein when the wax precipitation prediction indicates a gelation risk threshold will be exceeded during the selected time period for the new batch of crude oil, the gelation analytics tool recommends the at least one shutdown is modified to reduce a likelihood of gelation occurring for the new batch of crude oil in the pipeline.

14. The computer-implemented method of claim 12, wherein the gelation analytics tool determines a position of the new batch of crude oil in the pipeline based upon a flow rate of the new batch of crude oil.

15. The computer-implemented method of claim 12, wherein the new batch of crude oil is a mixture of a first batch of crude oil and a second batch of crude oil and wherein the gelation analytics tool uses flow rate data to determine a proportion of the first batch of crude oil and a proportion of the second batch of crude oil in the new batch of crude oil.

16. The computer-implemented method of claim 15,
wherein the first batch of crude oil has a first wax component comparable to data upon which the first wax precipitation model is based; and
wherein the second batch of crude oil has a second wax component comparable to data upon which the second wax precipitation model is based.

17. The computer-implemented method of claim 12, wherein the forecasted ambient conditions include one or more of forecasted weather, forecasted soil conditions, and forecasted water temperatures.

18. A computer-readable medium comprising instructions that when executed by a processor perform a method comprising:
providing, by a gelation analytics tool, a wax precipitation model of a percentage of wax precipitation over a temperature range for a batch of crude oil, the wax precipitation model based upon thermodynamic behavior of wax in the batch of crude oil;
providing, by the gelation analytics tool, a pipeline temperature model of predicted temperatures along the pipeline over a selected time period, the pipeline temperature model based upon forecasted ambient conditions about the pipeline during the selected time period;
receiving a proposed schedule comprising at least one shutdown of the pipeline during the selected time period for a new batch of crude oil;
generating, by the gelation analytics tool, a wax precipitation prediction for the new batch of crude oil during the selected time period by applying the pipeline temperature model during the selected time period to the wax precipitation model; and
outputting, by the gelation analytics tool, the wax precipitation prediction for managing the new batch of crude oil in the pipeline.

19. The computer-readable medium of claim 18, wherein when the wax precipitation prediction indicates a gelation risk threshold will be exceeded during the selected time period for the new batch of crude oil, the gelation analytics tool recommends the at least one shutdown is modified to reduce a likelihood of gelation occurring for the new batch of crude oil in the pipeline.

20. The computer-readable medium of claim 18, wherein the new batch of crude oil is a mixture of a first batch of crude oil and a second batch of crude oil and wherein the gelation analytics tool uses flow rate data to determine a proportion of the first batch of crude oil and a proportion of the second batch of crude oil in the new batch of crude oil.
